(19) United States
(12) Patent Application Publication
(10) Pub. No.: US 2017/0340505 A1
(43) Pub. Date: Nov. 30, 2017

(54) APPARATUS AND SYSTEMS FOR SYNCHRONIZING A USER TO AN EXOSKELETON

(71) Applicant: REWALK ROBOTICS LTD., Yokneam (IL)

(72) Inventor: LILACH HAVIDA, TZUR YITZHAK (IL)

(73) Assignee: REWALK ROBOTICS LTD., Yokneam (IL)

(21) Appl. No.: 15/610,263

(22) Filed: May 31, 2017

Related U.S. Application Data
(60) Provisional application No. 62/343,370, filed on May 31, 2016.

(51) Int. Cl. A61H 1/02; A61F 5/02

(52) U.S. Cl. CPC A61H 1/0292; A61F 5/02; A61H 2203/0406; A61H 2205/081; A61H 2203/0425

(57) ABSTRACT

Embodiments of an apparatus and systems for assisting mobility of a person comprise an exoskeleton device; and one or more straps extending between a first leg of the exoskeleton device and a second leg of the exoskeleton device, the one or more straps configured to reduce slumping of a user seated in the exoskeleton when the exoskeleton is in a sitting position.

### CROSS REFERENCE TO RELATED APPLICATIONS

This application claims priority to U.S. Provisional Patent Application No. 62/343,370, entitled “Apparatus and Systems for Synchronizing a User to an Exoskeleton,” filed May 31, 2016, the disclosure of which is incorporated by reference herein in its entirety.

### FIELD OF THE DISCLOSURE

Embodiments of the current disclosure are directed toward exoskeleton devices for providing gait/movement assistance, and more particularly, apparatus and systems for synchronizing a user to such devices.

### BACKGROUND

Various conditions contribute to the occurrence of disabilities in individuals that restrict or eliminate the individuals' capabilities for steady gait and/or movement, examples of which include neurological and physical injuries. Exoskeletons (“external skeletons”) have been used to allow such individuals regain some or all of their capabilities to stand and/or move about with little or no additional support despite their disabilities.

### SUMMARY OF SOME OF THE EMBODIMENTS

Some embodiments of an apparatus, method and systems for assisting the mobility of a person are disclosed herein. In some embodiments, a system for assisting the mobility of a person may comprise an exoskeleton device; and one or more straps extending between a first leg of the exoskeleton device and a second leg of the exoskeleton device, the one or more straps configured to reduce slumping of a user seated in the exoskeleton when the exoskeleton is in a sitting position. In some embodiments, the one or more straps may extend between a first vicinity of a first hip joint of the first leg of the exoskeleton to a second vicinity of a second hip joint of the second leg of the exoskeleton. In some embodiments, the one or more straps can include an elastic material. In some embodiments, the one or more straps can be detachable from the exoskeleton, while in others, the one or more straps may be fixedly connected to the exoskeleton. In some embodiments, the one or more straps can be movably connected to the exoskeleton.

In some embodiments, the one or more straps may be connected to the exoskeleton via a hook-and-eye locking mechanism. In some embodiments, the one or more straps may be connected to the exoskeleton via a magnetic attachment. In some embodiments, the one or more straps may include one or more of a fabric, a polymer and a plastic material.

Some embodiments of the current disclosure may include a method for assisting mobility of a person, the method comprising the steps of lowering an exoskeleton device to a sitting position or raising the exoskeleton device to a standing position, and arranging one or more straps to extend between a first leg of the exoskeleton device and a second leg of the exoskeleton device such that the one or more straps provide support to a user of the exoskeleton device to reduce slumping of the user in the exoskeleton device upon the lowering or the raising of the exoskeleton device. In some embodiments, the one or more straps extend between a first vicinity of a first hip joint of the first leg of the exoskeleton to a second vicinity of a second hip joint of the second leg of the exoskeleton. In some embodiments, the one or more straps can include an elastic material.

In some embodiments, the one or more straps may be detachable from the exoskeleton, while in yet others, the one or more straps can be fixedly connected to the exoskeleton. In some embodiments, the one or more straps may be movably connected to the exoskeleton. In some embodiments, the one or more straps may be connected to the exoskeleton via a hook-and-eye locking mechanism. And in some embodiments, the one or more straps can be connected to the exoskeleton via a magnetic attachment. In some embodiments, the one or more straps may include one or more of a fabric, a polymer and a plastic material.

Some embodiments of the current disclosure include a method for assisting mobility of a person, such method comprising the steps of determining a center of gravity of a user of an exoskeleton device as the exoskeleton device transitions between a sitting position and a standing position; and arranging a strap to extend between a first leg of the exoskeleton device and a second leg of the exoskeleton device such that an axis of the center of gravity of the user intersects a surface of the strap when the exoskeleton device is not in the standing position. In some embodiments, the strap can be arranged to extend between the first leg and the second leg such that the axis of the center of gravity intersects the surface of the strap at least substantially continuously during the transition of the exoskeleton device between the sitting position and the standing position.

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein. It should also be appreciated that terminology explicitly employed herein that also may appear in any disclosure incorporated by reference should be accorded a meaning most consistent with the particular concepts disclosed herein.

### BRIEF DESCRIPTION OF THE DRAWINGS

The skilled artisan will understand that the drawings primarily are for illustrative purposes and are not intended to limit the scope of the inventive subject matter described herein. The drawings are not necessarily to scale; in some instances, various aspects of the inventive subject matter disclosed herein may be shown exaggerated or enlarged in the drawings to facilitate an understanding of different features. In the drawings, like reference characters generally refer to like features (e.g., functionally similar and/or structurally similar elements).

FIGS. 1A-C show several view of example illustrations of a strap attached to an exoskeleton for use in synchronizing a user to the exoskeleton, according to some embodiments.

FIGS. 2A-C show example illustrations of the operations of a strap attached to an exoskeleton for use in synchronizing a user to the exoskeleton as the user is rising up or sitting down, according to some embodiments.

### DETAILED DESCRIPTION OF SOME OF THE EMBODIMENTS

In some embodiments of the present disclosure, apparatus and systems including exoskeleton devices for providing gait/movement assistance, and more particularly, apparatus and systems for synchronizing a user to such devices are presented. Although amenable to various applications, specific embodiments are described herein, by way of example and not limitation, in order to illustrate the principles and features of the invention.

In some embodiments, an exoskeleton may be used to assist a user in moving about (translation, rotation, etc.), maintaining and/or attaining positions (e.g., standing up, sitting down, holding upright position, etc.), and/or the like, and the user may facilitate such activities with the use of mobility aids such as crutches, which may or may not be operably coordinated with the exoskeleton. Descriptions of several embodiments of an exoskeleton device and mobility aids for use with the exoskeleton devices are presented in one or more of the following publications, all of which are incorporated by reference herein in their entireties:
U.S. Pat. No. 7,153,242, issued Dec. 26, 2006, filed May 24, 2001, and entitled “Gait-locomotor apparatus;”
U.S. Pat. No. 8,905,955, issued Dec. 9, 2014, filed Jan. 7, 2013, and entitled “Locomotion assisting device and method;”
US Patent Publication No. 2012/0101415, published Apr. 26, 2012, filed Oct. 21, 2010, and entitled “Locomotion Assisting Apparatus with Integrated Tilt Sensor;”
US Patent Publication No. 2013/0253385, published Sep. 26, 2013, filed Mar. 21, 2012, and entitled “Motorized Exoskeleton Unit;”

US Patent Publication No. 2014/0005577, published Jan. 2, 2014, filed Jun. 28, 2012, and entitled “Airbag for Exoskeleton Device;” and

US Patent Publication No. 2014/0196757, published Jul. 17, 2014, filed Jan. 17, 2013, and entitled “Gait Device with a Crutch.”

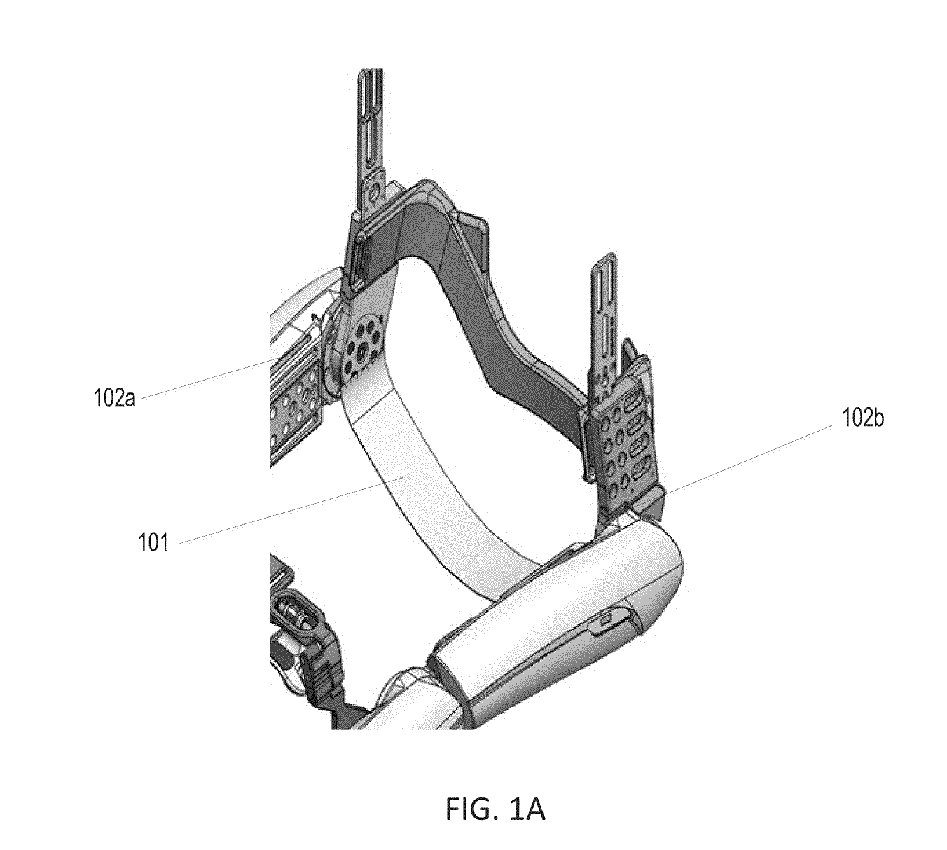

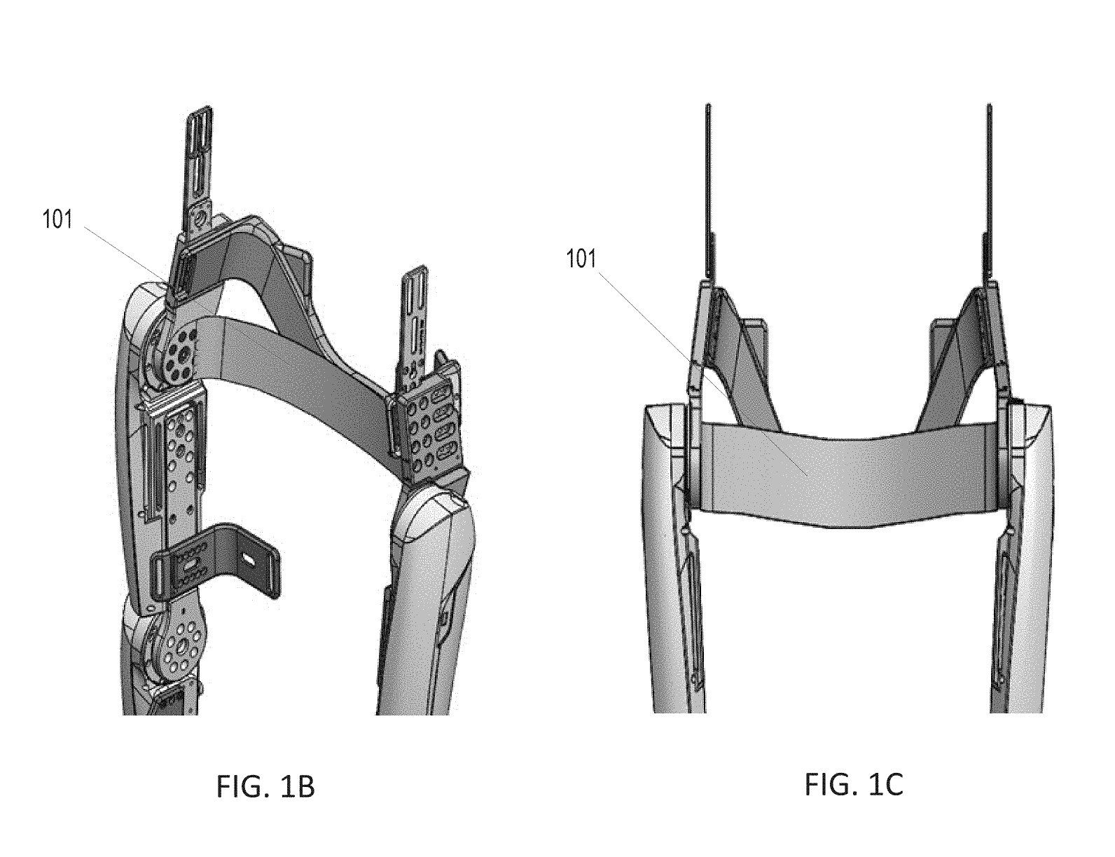

In some embodiments, the exoskeleton may comprise accessories that further facilitate the coordination of the exoskeleton with the user as the exoskeleton is operating. These accessories may provide support to a user when the exoskeleton is at rest and/or in motion, and also during transitional times when an exoskeleton is changing its state (e.g., standing up, sitting down, etc.). The accessories may be integrally attached to the exoskeleton or may be detachable. Examples of accessories that may be included in an exoskeleton device include a strap that, in some embodiments, enhances exoskeleton-user coordination or synchronicity. For example, when an exoskeleton is changing its state (e.g., standing up, sitting down, etc.), there may be a lag before the user reacts to the change (even when the user is aware of the coming change (e.g., due to a notice or alert from the exoskeleton)). In such embodiments, a strap 101 may support the user and facilitate the synchronization of the exoskeleton's movements with that of the user's. For example, with reference to FIG. 1A, a strap 101 may run across under a user's backside when a user is sitting down while using the exoskeleton device. Such a strap may provide support to the user when the exoskeleton and the user are at rest in a sitting position. Further, when the exoskeleton is standing up, the strap 101 may provide an upward support to the buttocks/thighs of the user, assisting the user in standing up and increasing the user/exoskeleton synchronization or coordination. When the exoskeleton is lowering down to assume a sitting position, similarly, the strap 101 may provide support to the buttocks/thighs of the user so as to minimize or avoid slumping by the user. In some embodiments, after the user has sat down and the exoskeleton is at rest, the strap 101 may also provide support to the seated user. In some embodiments, the positioning of the strap 101 with respect to the exoskeleton may be configured in such a way that the center of gravity of a user of the exoskeleton aligns at least approximately with the middle section of the strap 101 when the exoskeleton is not in an erect position. In some embodiments, such alignment may occur continuously or substantially continuously as the exoskeleton transitions between sitting and erect positions.

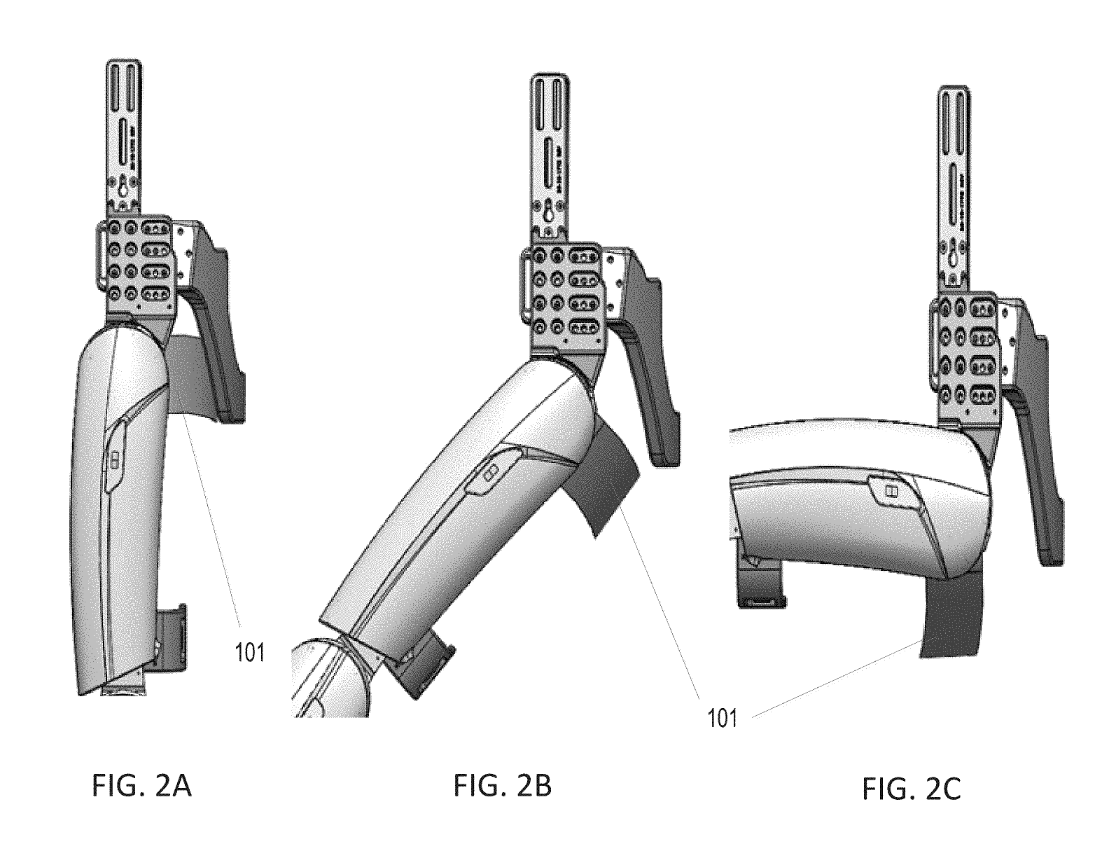

With reference to FIGS. 1B-C, in some embodiments, when the exoskeleton is in the standing position, the strap 101 may move outwards along with the exoskeleton to line behind the user in the vicinity of the user's lower back, buttocks and/or thighs. In some embodiments, the strap 101 may abut these limbs of the user. In any of these embodiments, the strap 101 is positioned behind the user in such a manner as to allow the user to comfortably stand without being pressured forward by the strap 101. FIGS. 2A-C show a progression of an exoskeleton going from a standing position to a sitting position. In the standing position (FIG. 2A), the strap 101 is located behind the user in the vicinity of the buttocks, for example, abutting the buttocks or lining up behind, but not abutting, the buttocks. During the transition towards the seating position (FIG. 2B), the strap 101 may start coming under the user, and may provide support to the backside of the user so that the user would not slump in the exoskeleton. Finally, in the seating position (FIG. 2C), the strap 101 is located under the user and provides support to the seating user. As such, in this manner, the strap 101 facilitates the synchronization or coordination of the exoskeleton and the user.

In some embodiments, similar analysis applies when the exoskeleton is going from a seated position to a standing position. When the exoskeleton starts to stand up from the seated position of FIG. 2C, the strap 101 assists the user in lifting up, thereby minimizing or avoiding slumping by the user. In some embodiments, the user may be utilizing a crutch in such embodiments, and as such may use the crutch to lift him/herself up. However, in some cases, there may be a lag between the exoskeleton lifting up and the user reacting to the lift up, even when the user is aware of the coming movement of the exoskeleton. In such situations, the strap 101 contributes to the reduction or avoidance of any slump or lagging by the user by providing an upward force to lift the user up in synchrony along with the exoskeleton.

In some embodiments, the strap 101 may be sized and shaped in any way so as to provide comfort and support to the user when in use. For example, the strap 101 may be a strip running from one hip joint 102a of the exoskeleton to another hip joint 102b. The strap 101 may be rectangular in shape and may have a width of about 1 ft., about half foot, about quarter foot, about 0.1 ft., including all values and sub ranges in between. In some embodiments, the shape may be such that the strap 101 is wider towards the middle and narrower towards the ends, thereby providing a wider surface area to support the buttocks of the user and narrower points of contact at the ends for use, for example, in fastening the strap to the exoskeletons. In some embodiments, the strap 101 may be curved so as to substantially follow (i.e., closely follow or mimic) the curvature of the limbs of the user when the strap 101 abuts the limbs (e.g., thighs and buttocks). Further, the curvature of the strap 101 may be such that when the exoskeleton is in an erect position, the strap 101 provides room to the user to comfortably stand without being pressure forward by the strap 101. For example, the strap 101 may be gently abutting the back surface of the user, or the trap 101 may not be abutting but may be generally in the vicinity of the back surface. In some embodiments, the strap may be designed so as to incorporate features that enhance the aesthetics and/or functions of the strap. For example, the strap may include openings (one or more distributed over the strap) that allow for circulation of air.

As mentioned above, in some embodiments, the strap 101 may be detachable from the exoskeleton or it may be integrally or fixedly connected to it. For example, the strap 101 may be designed so that a connection element (not shown) can be used to connect the strap 101 to the exoskeleton device 101. The connection element can be a structure independent of both the strap 101 and the exoskeleton device, or it may be part of the strap 101 and/or the exoskeleton. As an example, the connection element could be a projection (e.g., screw) that could be inserted into hollow spaces (bores) of the strap 101 and a part of the exoskeleton so that the strap 101 is connected to the exoskeleton. In some embodiments, the connection element may be an element attached to the exoskeleton and/or the strap 101 that locks into the strap 101 and/or the exoskeleton, respectively. For example, the connection element may be an element on the strap 101 that includes a “hook” or an “eye” in a hook-and-eye closure type locking mechanism, and the element may lock into the “eye” or “hook,” respectively, included in the exoskeleton. In general, a connection mechanism that establishes a secure connection between the strap 101 and an exoskeleton so that the strap 101 could provide support and comfort to the user can be used in attaching the strap 101 to the exoskeleton. In some embodiments, the strap 101 may be integrally (i.e., monolithically) and/or seamlessly attached to the exoskeleton. In some embodiments, the connections between the strap 101 and the exoskeleton may be magnetic in nature.

In some embodiments, the ends of the strap 101 may be fixedly or movably connected to parts of the exoskeleton. For examples, the ends may be fixedly or movably connected to the exoskeleton in the vicinity of the hip joints 102a and 102b. By “vicinity of a hip joint”, in some embodiments, it may be understood to mean within about 1 ft., about 0.5ft., about 0.25 ft., of a hip joint of the exoskeleton, including all values and sub ranges in between. In some embodiments, the ends of the strap 101 may be connected to the “thighs” of the exoskeleton, in addition to or instead of the hip joints 102a and 102b of the exoskeleton. For example, an end of the strap 101 may be positioned anywhere between the hip joint 102a (or 102b) and a knee joint (on the same leg) of the exoskeleton, allowing for the strap 101 to extend between the two legs of the exoskeleton anywhere along the thighs of the exoskeleton. In some embodiments, the connections between the strap and the exoskeleton may be movable, allowing for the adjustment of the strap 101 once the strap 101 is installed. The term “movable” includes the capability to change position rotationally, translationally, orientationally, and/or the like. For example, the connections may be movable within the range described above for the “vicinity of the hip joint.”

In some embodiments, the strap 101 may be made from any one or more of plastics, polymers, fabrics, etc. The strap 101 may have any number of properties that lend to its use as a support mechanism in an exoskeleton device. For example, the strap 101 may be elastic or flexible enough to be comfortable when a user sits into it, but sturdy enough to provide enough support so that the user is not slumping when seated (in particular, when the exoskeleton is in the process of assuming the seated position). The strap 101 may also be padded and/or insulated to enhance the comfort and/or functionality of the strap 101, while providing, for example, opening to allow circulation of air.

In some embodiments, the strap 101 may comprise a plurality of straps. For example, the strap 101 may comprise two, three, four, five, six, seven, eight, etc., straps spaced from each other in a regular or irregular manner. In some embodiments, two or more of the plurality of straps may be attached or connected to the exoskeleton at the same attachment point (for example, in the manner described above with respect to a single strap). In some embodiments, one or the plurality of straps may be in the form of a mesh.

While various inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, components and configurations described herein are meant to be an example and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure. Still further, some embodiments disclosed herein are distinguishable over prior art references by specifically lacking one or more features disclosed in the prior art; that is, claims to such embodiments may include negative limitations so as to be distinguished from the prior art.

Also, various inventive concepts may be embodied as one or more methods, of which an example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

Any and all references to publications or other documents, including but not limited to, patents, patent applications, articles, webpages, books, etc., presented anywhere in the present application, are herein incorporated by reference in their entirety. Moreover, all definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of” “only one of” or “exactly one of” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

1. A system for assisting mobility of a person, comprising:
an exoskeleton device; and
one or more straps extending between a first leg of the exoskeleton device and a second leg of the exoskeleton device, the one or more straps configured to reduce slumping of a user seated in the exoskeleton when the exoskeleton is in a sitting position.

2. The system of claim 1, wherein the one or more straps extend between a first vicinity of a first hip joint of the first leg of the exoskeleton to a second vicinity of a second hip joint of the second leg of the exoskeleton.

3. The system of claim 1, wherein the one or more straps include an elastic material.

4. The system of claim 1, wherein the one or more straps are detachable from the exoskeleton.

5. The system of claim 1, wherein the one or more straps are fixedly connected to the exoskeleton.

6. The system of claim 1, wherein the one or more straps are movably connected to the exoskeleton.

7. The system of claim 1, wherein the one or more straps are connected to the exoskeleton via a hook-and-eye locking mechanism.

8. The system of claim 1, wherein the one or more straps are connected to the exoskeleton via a magnetic attachment.

9. The system of claim 1, wherein the one or more straps include one or more of a fabric, a polymer and a plastic material.

10. A method for assisting mobility of a person, comprising:
lowering an exoskeleton device to a sitting position or raising the exoskeleton device to a standing position, and
arranging one or more straps to extend between a first leg of the exoskeleton device and a second leg of the exoskeleton device such that the one or more straps provide support to a user of the exoskeleton device to reduce slumping of the user in the exoskeleton device upon the lowering or the raising of the exoskeleton device.

11. The method of claim 10, wherein the one or more straps extend between a first vicinity of a first hip joint of the first leg of the exoskeleton to a second vicinity of a second hip joint of the second leg of the exoskeleton.

12. The method of claim 10, wherein the one or more straps include an elastic material.

13. The method of claim 10, wherein the one or more straps are detachable from the exoskeleton.

14. The method of claim 10, wherein the one or more straps are fixedly connected to the exoskeleton.

15. The method of claim 10, wherein the one or more straps are movably connected to the exoskeleton.

16. The method of claim 10, wherein the one or more straps are connected to the exoskeleton via a hook-and-eye locking mechanism.

17. The method of claim 10, wherein the one or more straps are connected to the exoskeleton via a magnetic attachment.

18. The method of claim 10, wherein the one or more straps include one or more of a fabric, a polymer and a plastic material.

19. A method for assisting mobility of a person, comprising:
determining a center of gravity of a user of an exoskeleton device as the exoskeleton device transitions between a sitting position and a standing position; and
arranging a strap to extend between a first leg of the exoskeleton device and a second leg of the exoskeleton device such that an axis of the center of gravity of the user intersects a surface of the strap when the exoskeleton device is not in the standing position.

20. The method of claim 19, wherein the strap is arranged to extend between the first leg and the second leg such that the axis of the center of gravity intersects the surface of the strap at least substantially continuously during the transition of the exoskeleton device between the sitting position and the standing position.
